(19) United States
(12) Patent Application Publication
(10) Pub. No.: US 2026/0232981 A1
(43) Pub. Date: Aug. 13, 2026

(54) SCRUBBING AND DISINFECTING A NEEDLELESS CONNECTOR HUB

(71) Applicant: Alera Medtech Inc., Oakville (CA)

(72) Inventor: Robert F. Rioux, Ashland, MA (US)

(21) Appl. No.: 19/534,673

(22) Filed: Feb. 9, 2026

Related U.S. Application Data
(60) Provisional application No. 63/755,686, filed on Feb. 7, 2025.

(51) Int. Cl. A61M 39/16; A61M 25/00

(52) U.S. Cl. CPC A61M 39/16; A61M 25/0014; A61M 2209/10

(57) ABSTRACT

The present invention generally relates to cleaning and disinfecting of medical devices, and, more particularly, to devices and assemblies for use in connection with medical procedures that involve or require the disinfection of one or more needleless connector hubs.

### CROSS-REFERENCE TO RELATED APPLICATIONS

This application claims priority to, and the benefit of, U.S. Provisional Application No. 63/755,686, filed on Feb. 7, 2025, the content of which is incorporated by reference herein in its entirety.

### TECHNICAL FIELD

The present invention generally relates to cleaning and disinfecting of medical devices, and, more particularly, to devices and assemblies for use in connection with medical procedures that involve or require the disinfection of one or more needleless connector hubs.

### BACKGROUND

A needleless connector (NC) is used on most if not all intravascular devices. An NC provides an easy access point for infusion connection. Colonization of an NC can result in an infection in the body of a patient, and such colonization can be the cause of many or most postinsertion catheter-related infections. If an NC is not properly disinfected, contamination and subsequent biofilm formation within the NC and catheters will increase the potential for infecting the patient.

### SUMMARY

The invention of the present disclosure provides devices and assemblies for providing scrubbing and/or disinfecting of one or more needleless connector hubs. Accordingly, a catheter-related bloodstream infection (CRBI) in a patient can be eliminated, or at least the likelihood of such an infection can be reduced, by a medical professional using a device or assembly consistent with the present disclosure, rather than using a common prepackaged alcohol disinfection pad or swab, to scrub and disinfect the needleless connector (NC) hub.

In particular, in one embodiment consistent with the present disclosure, a flush syringe assembly is provided, which includes a syringe (which can be preloaded/prefilled with a saline flush solution) and a disinfection unit removably attached to a distal tip of the syringe. The disinfection unit, which may be in the form of a container, can have a removable lid or cover, such as a piece of foil, that can be peeled from the top of the disinfection unit to reveal the inside of the disinfection unit. Upon removing the cover, an NC hub can then be inserted into the inside of the disinfection unit to both scrub and disinfect the NC hub. In particular, the inside of the disinfection unit includes one or more disinfecting agents (such as isopropyl alcohol) as well as an assembly of flexible bristles or other flexible extending elements that can physically contact and mechanically clean the NC hub when the hub is placed and moved within the disinfection unit.

After the NC hub has been rotated, or otherwise moved sufficiently inside the disinfection unit to clean and disinfect the NC hub by mechanical contact cleaning (bristles/elements) and chemical disinfection (disinfecting agent or agents), the NC hub may be removed from the disinfection unit, and the disinfection unit then can be removed from the tip of the syringe by, for example, unscrewing the disinfection unit from the tip of the syringe. For example, the disinfection unit may include a Luer-like connection at a base portion thereof for releasably coupling to a corresponding Luer-like connection at a distal tip of the syringe. Upon removal of the disinfection unit, the tip of the syringe then can be attached to the NC hub, and the saline in the syringe can be pushed out of the syringe and through the NC to thereby flush the system.

In some embodiments, the base portion of the disinfection unit may be hollow, such that, when coupled to the syringe, the hollow base portion, and thus the interior of the disinfection unit, is in fluid communication with the distal tip of the syringe such that the syringe may be used to flush out the disinfection unit. In particular, in this embodiment, a slit valve disk may be positioned within the disinfection unit, adjacent to the base portion, and positioned between an interior surface of the disinfection unit (at the base portion) and the assembly of bristles (which include an aperture formed within a portion thereof). While the disinfection unit remains coupled to the syringe, and upon sufficient application of force being applied to the syringe plunger, fluid is expelled from the syringe and through the slit valve disk and into the disinfection unit top thereby flush out the interior of the disinfection unit.

Yet still, in another embodiment consistent with the present disclosure, a standalone disinfection unit is provided. In particular, rather than being releasably coupled to a syringe, the disinfection unit is a single-use, stand-alone device, which includes a container filled with one or more disinfecting agents (such as isopropyl alcohol) and further including an assembly of flexible bristles or other flexible extending elements that can physically contact and mechanically clean the NC hub when the hub is placed and moved within the disinfection unit. Rather than having a Luer-like connector at its base, the disinfection unit may include a stem-like base with protrusions, generally in the form of wings, extending from the base to thereby aid a user in holding and twisting the disinfection unit during use.

Accordingly, the inventive device thus provides an all-in-one solution that simplifies the disinfection process, and the device does so by using a prefilled saline flush syringe with a disinfection unit that is removably attached to a tip of the syringe and that has within it one or more disinfecting agents as well as a plurality of extending elements for physically contacting and mechanically cleaning a needleless connector hub that is placed inside the disinfection unit and moved within the disinfection unit.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a perspective view showing an exemplary embodiment of a flush syringe assembly consistent with the present disclosure.

FIG. 2 is an enlarged perspective view of a disinfection unit, illustrating removal of the cover or lid therefrom.

FIG. 3 is an exploded view of one embodiment of a disinfection unit, in which the unit includes a container or cap member having an integral body, an annular wall extending from a closed proximal or base end to an open distal end cooperatively defining a chamber or cavity within.

FIG. 4 is an enlarged perspective view of a bristle assembly of the disinfection unit.

FIG. 5 is an exploded view of the disinfection unit of FIG. 3, illustrating the bristle assembly in a compressed configuration for insertion into the container.

FIGS. 6 and 7 are sectional and perspective, partly in section, views, respectively, of the disinfection unit of FIG. 3, illustrating the various components thereof.

FIG. 8 is an exploded view of another embodiment of a disinfection unit, in which the unit includes a container or cap member having an integral body, an annular wall extending from an open proximal or base end to an open distal end cooperatively defining a chamber or cavity within. The open base end allows for the interior of the container to be in fluid communication with the distal tip of the syringe such that the syringe may be used to flush out the disinfection unit during use.

FIGS. 9 and 10 are sectional and perspective, partly in section, views, respectively, of the disinfection unit of FIG. 8, illustrating the various components thereof.

FIG. 11 is a perspective view illustrating a needleless connector (NC) hub to be inserted into the inside of the disinfection unit to both scrub and disinfect the NC hub.

FIG. 12 is a perspective view showing another embodiment of a disinfection unit that is a single-use, stand-alone device (i.e., separate from a flushing syringe).

FIG. 13 is an exploded view of the disinfection unit of FIG. 12.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure relates generally to cleaning and disinfecting of medical devices, and, more particularly, to devices and assemblies for use in connection with medical procedures that involve or require the disinfection of one or more needleless connector hubs.

The invention of the present disclosure provides devices and assemblies for providing scrubbing and/or disinfecting of one or more needleless connector hubs. Accordingly, a catheter-related bloodstream infection (CRBI) in a patient can be eliminated, or at least the likelihood of such an infection can be reduced, by a medical professional using a device or assembly consistent with the present disclosure, rather than using a common prepackaged alcohol disinfection pad or swab, to scrub and disinfect the needleless connector (NC) hub.

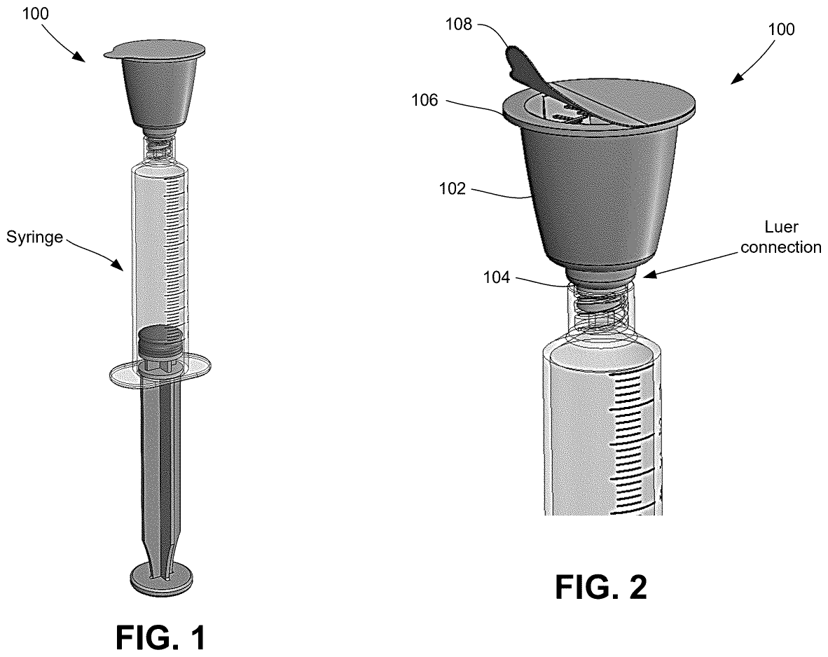

FIG. 1 is a perspective view showing an exemplary embodiment of a flush syringe assembly consistent with the present disclosure, which includes a syringe (which can be preloaded/prefilled with a saline flush solution) and a disinfection unit 100 removably attached to a distal tip of the syringe, generally via a Luer connection or similar connection known in the art. For example, the disinfection unit may include a male Luer connector portion that connects to a corresponding female Luer lock connector portion of the distal tip of the syringe. The assembly may be packaged in a Tyvek pouch with saline prefilled in the syringe, and a cleaning agent, such as isopropyl alcohol, provided in the disinfection unit.

The syringe may generally include a barrel including a cylindrical side wall having an inside surface defining a chamber for retaining fluid, an open proximal end, and a distal end including a distal wall with a tip extending distally therefrom and having a passageway therethrough in fluid communication with the chamber. The syringe further include a plunger including an elongate body portion having a proximal end, a distal end, and a stopper slidably positioned in fluid-tight engagement with the inside surface of the barrel for drawing fluid into and driving fluid out of the chamber by movement of the stopper relative to the barrel, the elongate body portion extending outwardly from the open proximal end of the barrel. It should be noted that the syringe may be a common polypropylene syringe filled with a saline solution (e.g., 0.9% saline), for example.

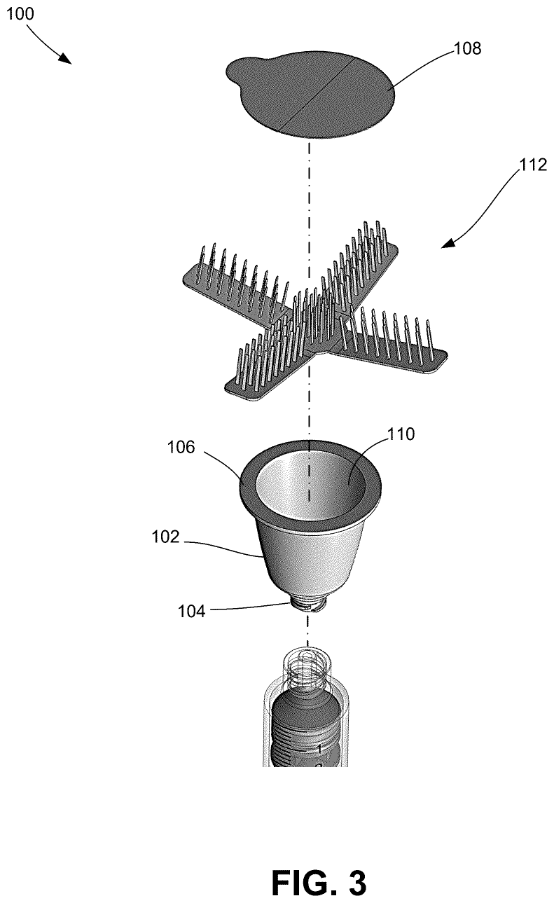

FIG. 2 is an enlarged perspective view of the disinfection unit 100. As shown, the disinfection unit is generally in the form of a container or cap having an integral body 102, an annular wall extending from a proximal end 104 (which can be coupled to the distal tip of the syringe) to an open end 106, in which a chamber or inner cavity 110 is defined within. The unit 100 further includes a user-peelable lid or cover 108 sealed to the open end 106 of the container to thereby contain a fluid within the inner cavity 110, which may generally include a cleaning agent, such as isopropyl alcohol. The lid or cover 108 effectively maintains a seal with the container, effectively preventing the cleaning agent from leaking from the container. The lid or cover 108 can be thermally-, ultrasonically-, or RF-welded to the top rim of the container. For example, the lid or cover 108 be a foil top with a polymer bottom surface thereby enabling thermal sealing.

### “Closed” Disinfection Unit

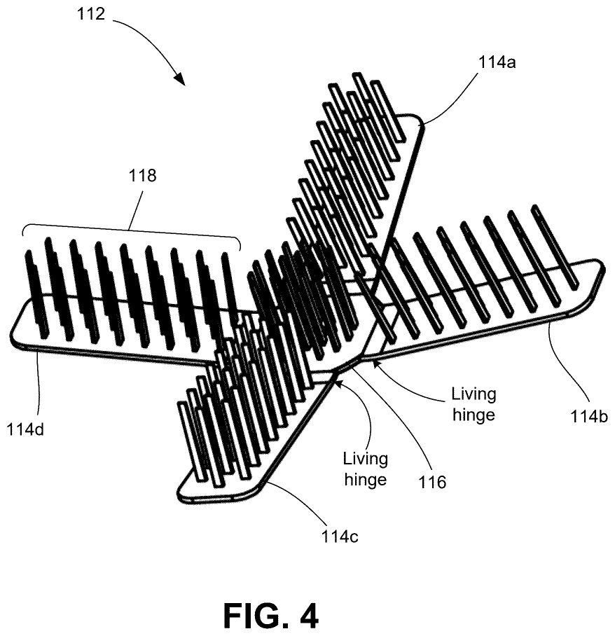

FIG. 3 is an exploded view of one embodiment of a disinfection unit 100, in which the unit includes a container or cap member having an integral body 102, an annular wall extending from a closed proximal or base end 104 to an open distal end 106 cooperatively defining a chamber or cavity within 110. The closed base end 104 includes a Luer connector portion for removably coupling the disinfection unit 100 to the distal tip of the syringe. In this “closed” configuration, there is no fluid exchange allowed between the syringe and the disinfection unit, such that fluid contamination between the syringe and container is prevented.

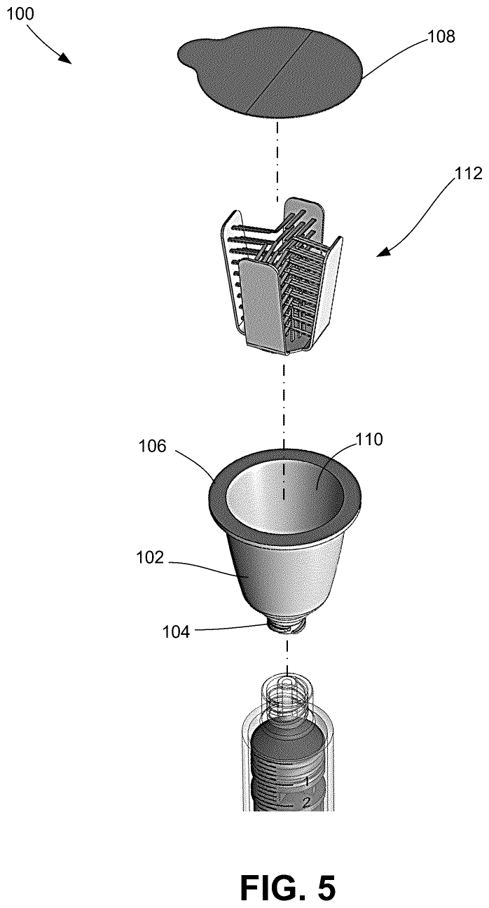

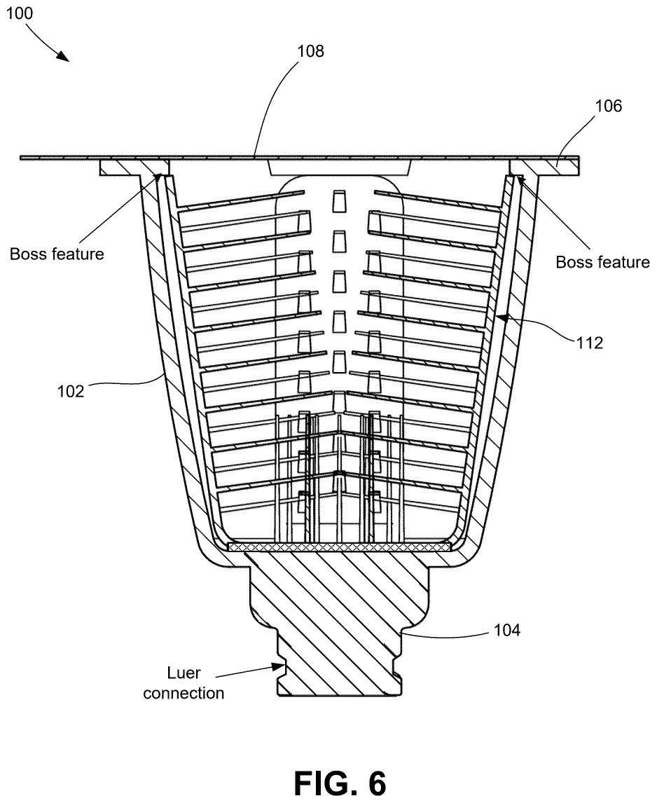

As shown, the disinfection unit 100 includes a bristle assembly 112 comprising a plurality of bristles configured to physically contact and mechanically clean an NC hub when the hub is placed and moved within the interior cavity 110 of the disinfection unit 100. For example, FIG. 4 is an enlarged perspective view of a bristle assembly 112. As shown, the bristle assembly 112 may be a thermoplastic molded one-piece component, comprising a plurality of arms 114a-114d extending from a center portion 116 via respective living hinges, wherein each arm 114a-114d includes an array 118 of flexible bristles that can mechanically scrape contaminants off of the NC hub during use. Each arm 114a-114d is configured to fold inwardly towards one another to form a compressed configuration so as to be received within the interior cavity 110, and maintain the compressed configuration during use. FIG. 5 is an exploded view of the disinfection unit of FIG. 3, illustrating the bristle assembly 112 in a compressed configuration for insertion into the container. The bristle assembly can be molded from various materials, including, but not limited to, low density polyethylene and/or polyurethane. Furthermore, coatings can be applied to enhance the wettability of the bristles, such as a hydrophilic coating applied to a low density polyethylene material. When compressed tightly, the bristles provide a superior scraping action to remove contaminants from the needle site to be cleaned and further provide enough surface tension to help hold cleaning agent within the container.

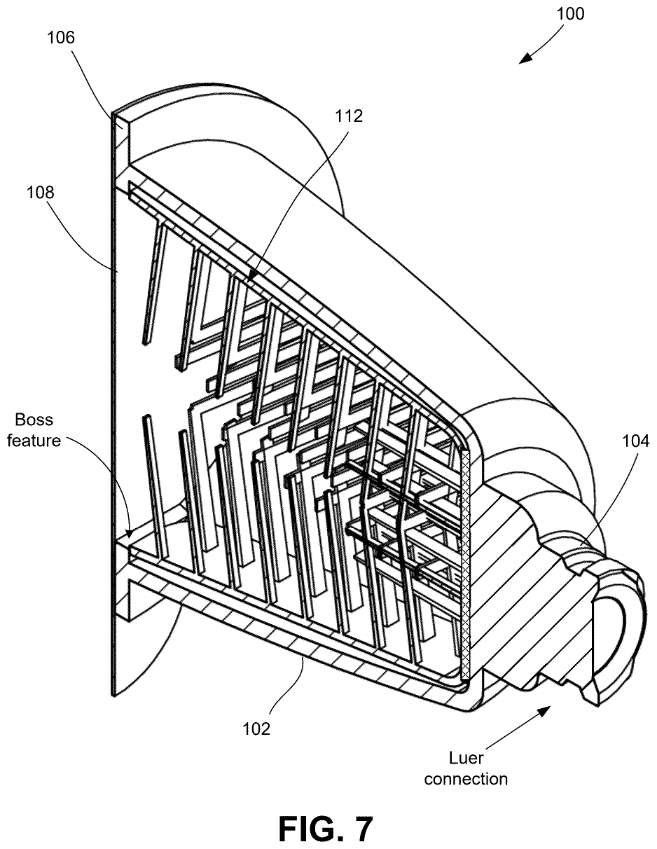

FIGS. 6 and 7 are sectional and perspective, partly in section, views, respectively, of the disinfection unit of FIG. 3, illustrating the various components thereof. As shown, a plurality of bosses may be disposed on the interior wall surface of the annular wall of the container body 102, the bosses protruding inwards from the interior wall surface and being adjacent to the open distal end 106 of the container. The arms 114a-114d of the bristle assembly 112 are held in place by the interior bosses.

### “Open” Disinfection Unit

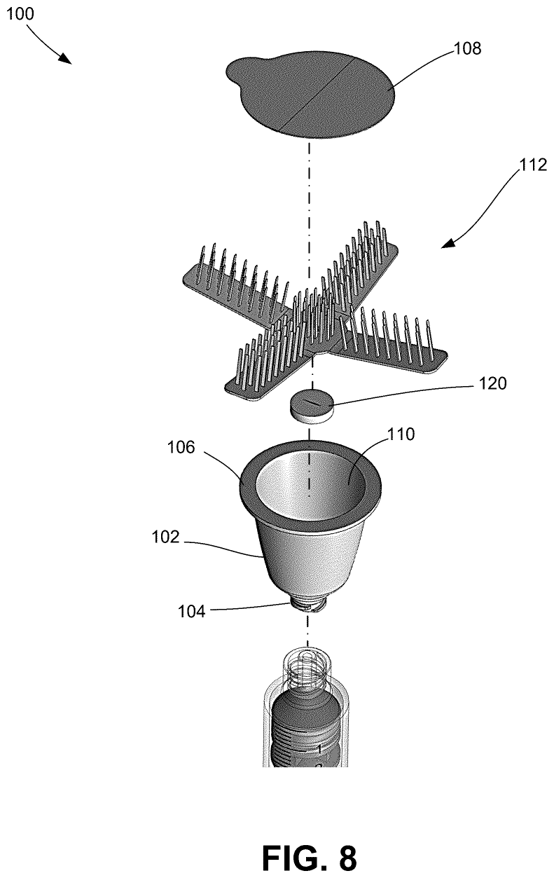

FIG. 8 is an exploded view of another embodiment of a disinfection unit 100, similar to the disinfection unit 100 of FIGS. 3-7, but includes an open proximal or base end 104 (as opposed to a closed proximal or base end), which allows for the interior of the container to be in fluid communication with the distal tip of the syringe such that the syringe may be used to flush out the disinfection unit during use.

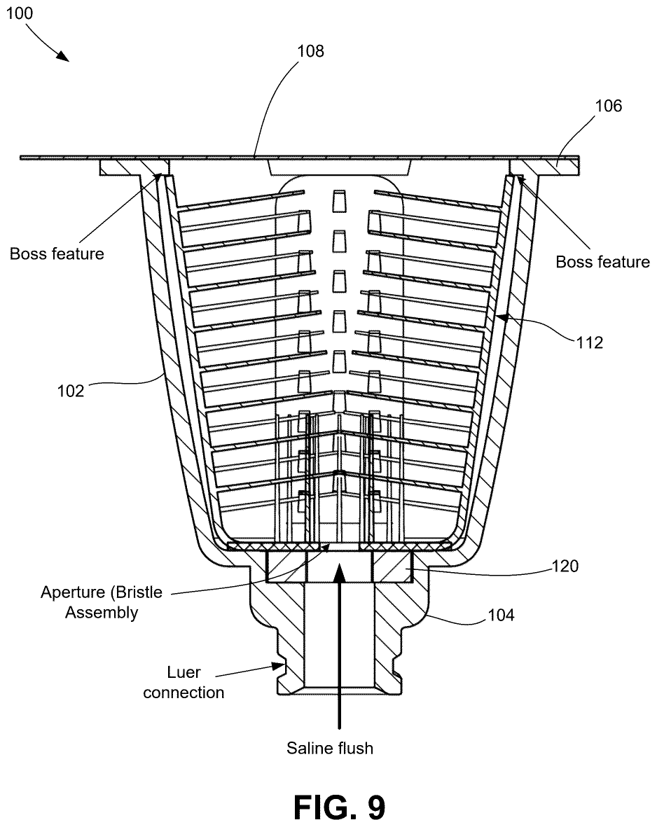

FIGS. 9 and 10 are sectional and perspective, partly in section, views, respectively, of the disinfection unit of FIG. 8, illustrating the various components thereof. In this embodiment, the unit 100 further includes a slit valve disk 120 positioned within the disinfection unit, adjacent to the base portion 104, and positioned between an interior surface of the disinfection unit (at the base portion 104) and the bristle assembly 112 (which includes an aperture formed within the center portion 116 thereof). While the disinfection unit 100 remains coupled to the syringe, and upon sufficient application of force being applied to the syringe plunger, fluid is expelled from the syringe and through the slit valve disk and into the disinfection unit top thereby flush out the interior of the disinfection unit.

### Scrubbing/Disinfecting of NC Hub

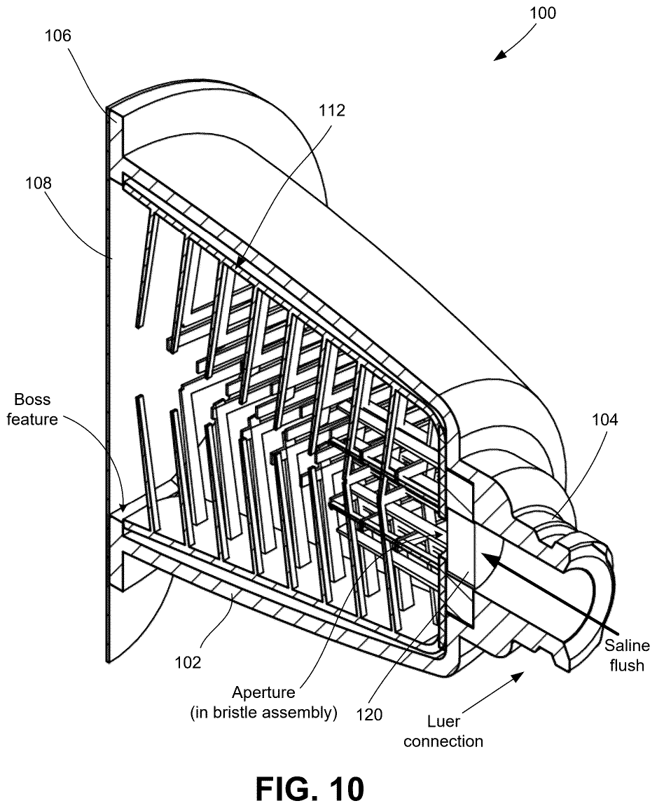

FIG. 11 is a perspective view illustrating a needleless connector (NC) hub to be inserted into the inside of the disinfection unit to both scrub and disinfect the NC hub. Upon removing the lid or cover from the disinfection unit, the NC hub can then be inserted into the inside of the disinfection unit to both scrub and disinfect the NC hub. In particular, the inside of the disinfection unit includes one or more disinfecting agents (such as isopropyl alcohol) as well as an assembly of flexible bristles or other flexible extending elements that can physically contact and mechanically clean the NC hub when the hub is placed and moved within the disinfection unit. After the NC hub has been rotated, or otherwise moved sufficiently inside the disinfection unit to clean and disinfect the NC hub by mechanical contact cleaning (bristles/elements) and chemical disinfection (disinfecting agent or agents), the NC hub may be removed from the disinfection unit, and the disinfection unit then can be removed from the tip of the syringe by, for example, unscrewing the disinfection unit from the tip of the syringe. For example, the disinfection unit may include a Luer-like connection at a base portion thereof for releasably coupling to a corresponding Luer-like connection at a distal tip of the syringe. Upon removal of the disinfection unit, the tip of the syringe then can be attached to the NC hub, and the saline in the syringe can be pushed out of the syringe and through the NC to thereby flush the system.

In some embodiments, the base portion of the disinfection unit may be hollow, such that, when coupled to the syringe, the hollow base portion, and thus the interior of the disinfection unit, is in fluid communication with the distal tip of the syringe such that the syringe may be used to flush out the disinfection unit. In particular, in this embodiment, a slit valve disk may be positioned within the disinfection unit, adjacent to the base portion, and positioned between an interior surface of the disinfection unit (at the base portion) and the assembly of bristles (which include an aperture formed within a portion thereof). While the disinfection unit remains coupled to the syringe, and upon sufficient application of force being applied to the syringe plunger, fluid is expelled from the syringe and through the slit valve disk and into the disinfection unit top thereby flush out the interior of the disinfection unit.

### “Stand-Alone” Disinfection Unit

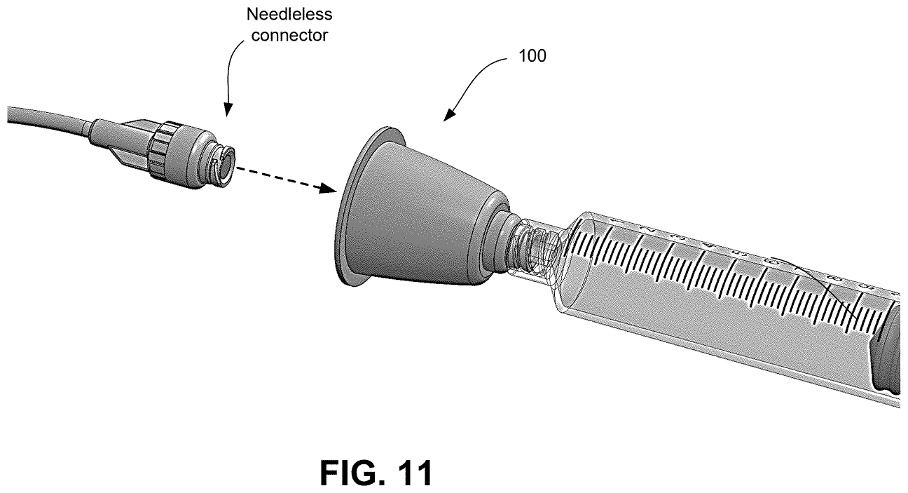

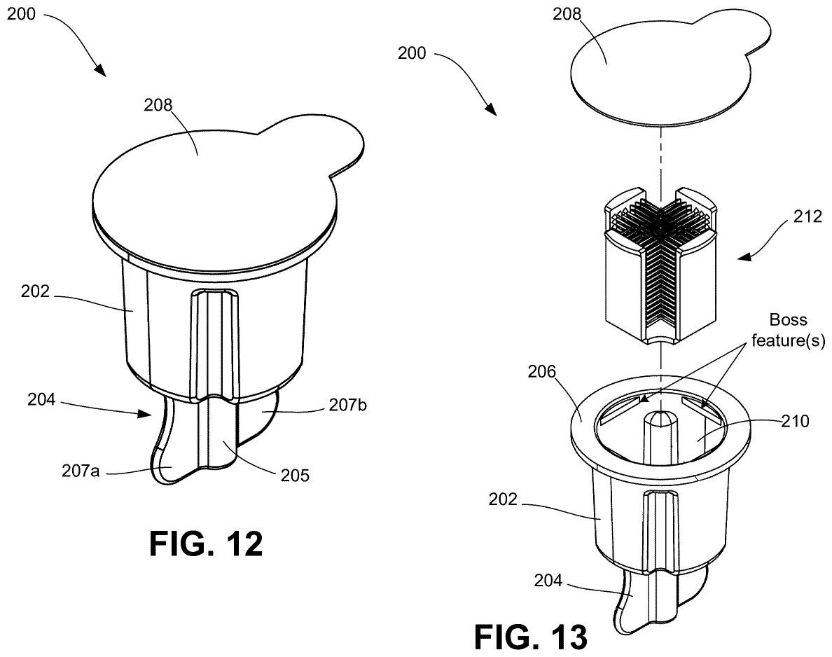

FIG. 12 is a perspective view showing another embodiment of a disinfection unit 200 that is a single-use, stand-alone device (i.e., separate from a flushing syringe). FIG. 13 is an exploded view of the disinfection unit 200. In particular, rather than being releasably coupled to a syringe, the disinfection unit 200 is a single-use, stand-alone device. As shown, the disinfection unit 200 is generally in the form of a container or cap having an integral body 202, an annular wall extending from a closed proximal end 204 to an open distal end 206, in which a chamber or inner cavity 210 is defined within. The unit 200 further includes a user-peelable lid or cover 208 sealed to the open end 206 of the container to thereby contain a fluid within the inner cavity 210, which may generally include a cleaning agent, such as isopropyl alcohol.

The disinfection unit 200 includes a bristle assembly 212 comprising a plurality of bristles configured to physically contact and mechanically clean an NC hub when the hub is placed and moved within the interior cavity 210 of the disinfection unit 200. As previously described herein, the bristle assembly 212 may be a thermoplastic molded one-piece component, comprising a plurality of arms extending from a center portion via respective living hinges, wherein each arm includes an array of flexible bristles that can mechanically scrape contaminants off of the NC hub during use. Each arm is configured to fold inwardly towards one another to form a compressed configuration so as to be received within the interior cavity 210, and maintain the compressed configuration during use. The bristle assembly 212 is held within the interior cavity 210 of the container via a plurality of bosses disposed on the interior wall surface of the annular wall of the container body 202.

In this embodiment, rather than having a Luer-like connector at its base, the disinfection unit 200 may include a stem-like base 205 with protrusions, generally in the form of wings 207a, 207b, extending from the stem portion 205 to thereby aid a user in holding and twisting the disinfection unit during use.

Accordingly, the inventive devices described herein thus provide an all-in-one solution that simplifies the disinfection process, and the device does so by using a prefilled saline flush syringe with a disinfection unit that is removably attached to a tip of the syringe and that has within it one or more disinfecting agents as well as a plurality of extending elements for physically contacting and mechanically cleaning a needleless connector hub that is placed inside the disinfection unit and moved within the disinfection unit.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A flush syringe assembly, comprising:
a barrel including a cylindrical side wall having an inside surface defining a chamber for retaining fluid, an open proximal end, and a distal end including a distal wall with a tip extending distally therefrom and having a passageway therethrough in fluid communication with the chamber;
a plunger including an elongate body portion having a proximal end, a distal end, and a stopper slidably positioned in fluid-tight engagement with the inside surface of the barrel for drawing fluid into and driving fluid out of the chamber by movement of the stopper relative to the barrel, the elongate body portion extending outwardly from the open proximal end of the barrel; and
a cap removably attached to the tip of the barrel, the cap having an opening to an inner cavity, the opening for receiving therethrough and the inner cavity for receiving therein at least a portion of a needleless access device, the cap containing at least one cleaning agent, the cap also containing a bristle component having a plurality of bristles.

2. The assembly of claim 1, wherein the cap includes a user-peelable seal covering the opening to the inner cavity of the cap.

3. The assembly of claim 1, wherein the seal is welded to a top rim of the cap that defines the opening of the cap.

4. The assembly of claim 3, wherein the seal is thermally, ultrasonically, or radiofrequency welded to the top rim of the cap.

5. The assembly of claim 1, wherein the bristle component comprises a thermoplastic molded one-piece component.

6. The assembly of claim 5, wherein the plurality of bristles of the thermoplastic molded one-piece bristle component extend into the inner cavity of the cap and are designed to physically contact and mechanically clean the portion of the needleless access device that is received through the opening and into the inner cavity of the cap.

7. The assembly of claim 1, wherein the cap also contains a slit valve disk.

8. The assembly of claim 1, wherein the at least one cleaning agent within the cap comprises isopropyl alcohol.
